(19) United States
(12) Patent Application Publication
(10) Pub. No.: US 2018/0360329 A1
(43) Pub. Date: Dec. 20, 2018

(54) PHYSIOLOGICAL SIGNAL SENSOR

(71) Applicant: YongLin Biotech Corp., New Taipei (TW)

(72) Inventors: CHIA-WEI CHANG, New Taipei (TW); CHENG-YAN GUO, New Taipei City (TW); YAO-HUNG KUO, New Taipei City (TW); JIANN-HUA WANG, New Taipei (TW); CHIH-HAO LIU, New Taipei (TW); YAO-TSUNG CHANG, New Taipei (TW); CHE-WEI CHANG, New Taipei (TW); DUN-YUN GAO, New Taipei (TW); MING-HSUN HSU, New Taipei (TW)

(21) Appl. No.: 15/989,298

(22) Filed: May 25, 2018

(30) Foreign Application Priority Data
May 27, 2017 (CN) 201710390473.0

(51) Int. Cl. A61B 5/024; A61B 5/0205; A61B 5/00; A61B 8/08

(52) U.S. Cl. CPC A61B 5/02405; A61B 5/0205; A61B 5/7257; A61B 5/14532; A61B 5/7225; A61B 5/02438; A61B 8/488

(57) ABSTRACT

A physiological signal sensor to measure life signs includes at least one first and one second Doppler detectors, at least one first amplification filter, at least one second amplification filter, a processor, and a transceiver. Data from the physiological signal sensor is sent to the first and second Doppler detectors to detect first and second physiological signals from different locations on a living body. The two physiological signals are sent to the first and second amplification filters and amplified and filtered, and converted to obtain a first and second digital sensed signal. Digital signal processing is carried out on the digital sensed signals to obtain first and second physiological information which are outputted via the transceiver.

### FIELD

The disclosure relates to a physiological signal sensor, and more particularly to a physiological signal sensor with more than one sensor therein.

### BACKGROUND

New semiconductor sensors are developed. The sensors provide specific sensing functionality such as image sensing, infrared sensing, ultrasonic sensing, thermal and humidity sensing, vibration sensing, Doppler sensing, physiological signal sensing, etc. and these are utilized in daily life. In the field of medical and health care, the sphygmomanometer, the blood glucose meter, and the pulse meter are products combining medicine and electronic techniques, and have the advantage such as being lightweight and/or small size. These products are portable, operable, and have low power-consumption.

However, the conventional measurement of heart-beat and/or respiration needs electrode pads adhered to the chest near the heart and/or the lungs and the electronic signals sensed by the electrode pads are processed by electronic device to obtain the heart rate and the respiration rate. The means of measurement needs long connection wires to connect the electrode pads and the electronic device, so the wires limit the movement of body and the movement of body influences the accuracy of measurement. Hence, the user typically lays down or sits until the end of the measurement. A user, especially the elder or a child, may feel sharp coldness at the moment the electrode pads are applied on his or her body. Accordingly, there is room for improvement within the art.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 illustrates a schematic of a physiological signal sensor in a first embodiment of the disclosure;

FIG. 2A to FIG. 2C are operational diagrams of the physiological signal sensor;

FIG. 3 illustrates a schematic of either a first Doppler detector or a second Doppler detector in the physiological signal sensor;

FIG. 4 illustrates an operational diagram of the physiological signal sensor in the disclosure;

FIG. 5A and FIG. 5B show operation flows of the physiological signal sensor;

FIG. 6 illustrates a schematic of a physiological signal sensor in a second embodiment of the disclosure;

FIG. 7A illustrates classifications of conventional gestures in a second embodiment of the disclosure;

FIG. 7B illustrates frequency domain signals corresponding to the classifications of conventional gestures in FIG. 7A;

FIG. 8 illustrates a schematic of a gesture and command mapping graphical user interface (GCM_GUI) in the second embodiment of the disclosure;

FIG. 9 illustrates a functional block diagram of a robot in the second embodiment of the disclosure;

FIG. 10 illustrates a flowchart of setting operations in the second embodiment of the disclosure;

FIG. 11 illustrates a flowchart of user operations in the second embodiment of the disclosure;

FIG. 12 illustrates a pipeline algorithm of a gesture recognition method in the disclosure;

FIG. 13 illustrates a schematic of a physiological signal sensor in a third embodiment of the disclosure;

FIG. 14 illustrates another example of the schematic of the physiological signal sensor in the third embodiment;

FIG. 15 illustrates a schematic of a Doppler detector in one embodiment of the disclosure;

FIG. 16 illustrates a schematic of a Doppler antenna in one embodiment of the disclosure;

FIG. 17A and FIG. 17B illustrate a flowchart of a method for heart-beat calculation in one embodiment of the disclosure;

FIG. 18 illustrates a flowchart of an amplitude normalization in one embodiment of the disclosure;

FIG. 19 illustrates a flowchart of a de-resonance method in one embodiment of the disclosure; and

FIG. 20 illustrates a flowchart of a method for respiration calculation in one embodiment of the disclosure.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein can be practiced without these specific details. In other instances, methods, procedures, and components have not been described in detail so as not to obscure the related relevant feature being described. Also, the description is not to be considered as limiting the scope of the exemplary embodiments described herein. The drawings are not necessarily to scale and the proportions of certain parts have been exaggerated to better illustrate details and features of the present disclosure.

Several definitions that apply throughout this disclosure will now be presented.

The term “substantially” is defined to be essentially conforming to the particular dimension, shape, or other feature that the term modifies, such that the component need not be exact. For example, “substantially cylindrical” means that the object resembles a cylinder, but can have one or more deviations from a true cylinder. The term “comprising,” when utilized, means “including, but not necessarily limited to”; it specifically indicates open-ended inclusion or membership in the so-described combination, group, series, and the like.

The present disclosure is described in relation to a physiological signal sensor.

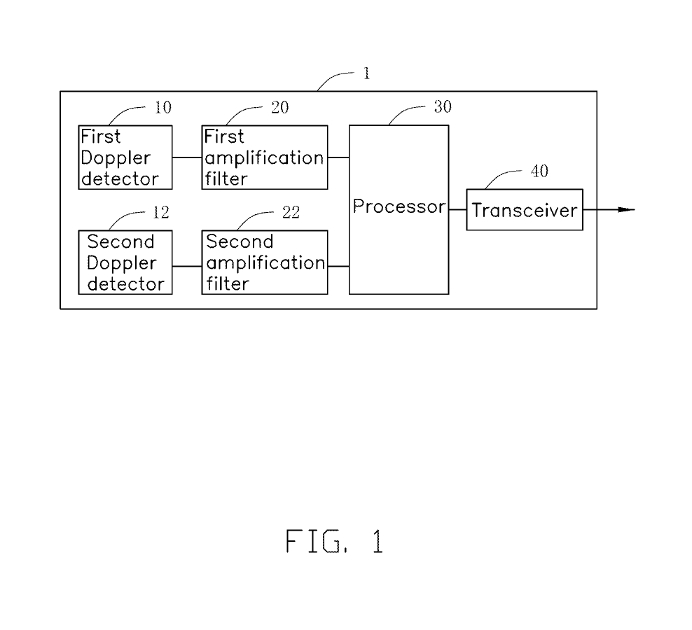

FIG. 1 shows a schematic of a physiological signal sensor in one embodiment of the disclosure. As shown in FIG. 1, a physiological signal sensor in the first embodiment of the disclosure includes at least one first Doppler detector 10, at least one second Doppler detector 12, at least one first amplification filter 20, at least one second amplification filter 22, a processor 30, and a transceiver 40. The physiological signal sensor is wearable such as around neck or on chest and configured to sense physiological information such as heart beat rate and respiration rate. The transceiver 40 is, for example, a wired transceiver or a wireless transceiver.

It should be noted that a number of the first Doppler detector 10, a number of the second Doppler detector 12, a number of the first amplification filter 20, and a number of the second amplification filter 22 are all arbitrary and can be arranged as necessary. In the embodiment, there are substantially at least two Doppler detectors and at least two amplification filters, and each amplification filter has a corresponding Doppler detector.

The first Doppler detector 10 is connected to the first amplification filter 20 and the second Doppler detector 12 is connected to the second amplification filter 22. The processor 30 is connected to the first amplification filter 20, the second amplification filter 22, and the transceiver 40. The first Doppler detector 10 detects a first physiological signal at a first location of the human body based on the Doppler effect and the second Doppler detector 12 detects a second physiological signal at a second location of the human body based on the Doppler effect. The first physiological signal and the second physiological signal are respectively sent to the first amplification filter 20 and the second amplification filter 22, and a first digital signal and a second digital signal are generated after the first physiological signal and the second physiological signal are amplified, filtered, and processed. The first digital sensed signal and the second sensed signal are then received and processed by the processor 30 so as to generate first physiological information and second physiological information, for ongoing transmission to the transceiver 40. The transceiver 40 sends out by wire or emits wirelessly the first physiological information and the second physiological information.

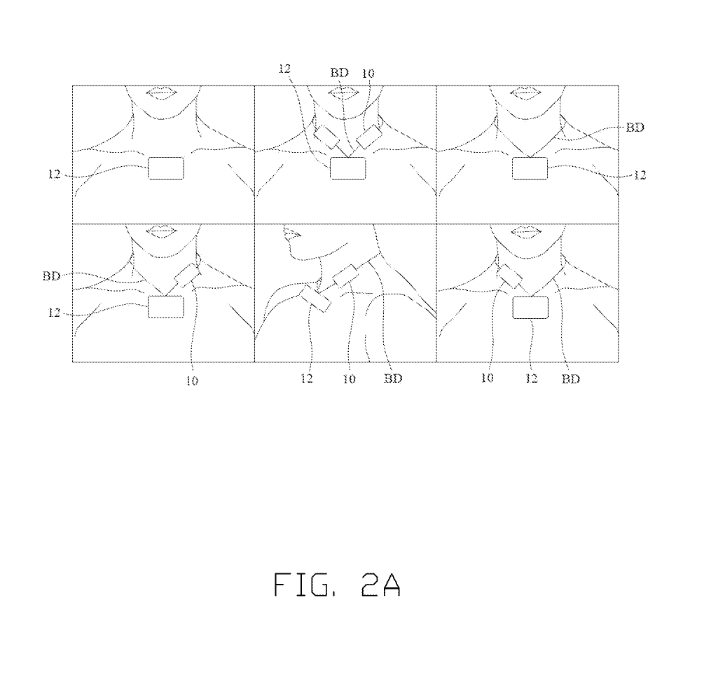

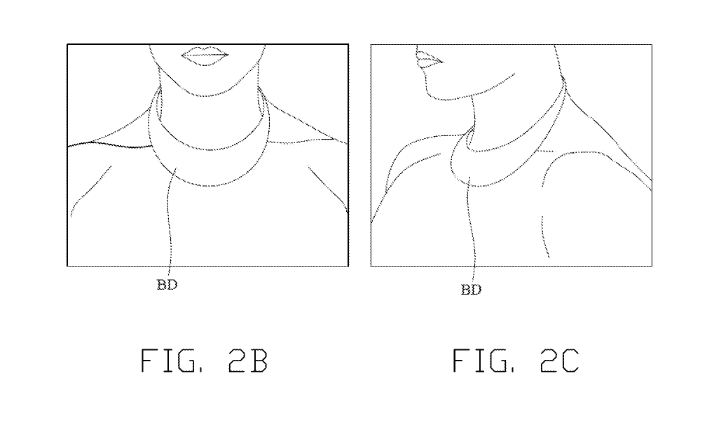

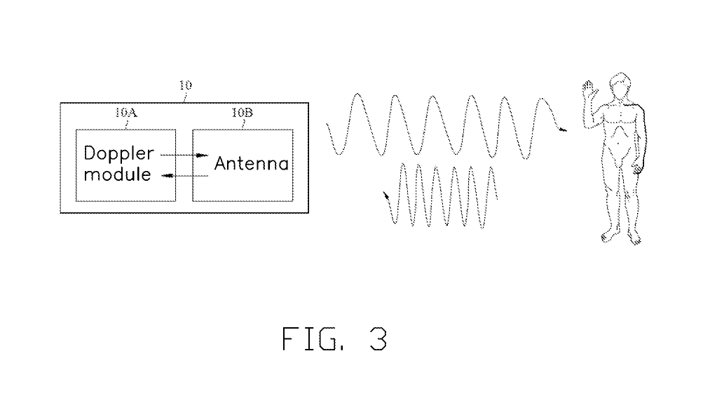

As shown in FIG. 2A, FIG. 2B, and FIG. 2C, the first Doppler detector 10 is arranged to be worn near an artery around the neck and is capable of obtaining heart beat related signal while the second Doppler detector 12 is arranged to be worn near the chest and the clavicle and is capable of obtaining signal related to respiration. In another embodiment, the first Doppler detector 10 and the second Doppler detector 12 are located on the stripe carrier BD of a necklace and arranged around artery near the neck or the clavicle.

The features of both the first Doppler detector 10 and the second Doppler detector 12 are similar. The first Doppler detector 10 and the second Doppler detector 12 have substantially identical or similar electric functionality. Taking the first Doppler 10 for example, the Doppler detector 10, as shown in FIG. 3, includes a Doppler module 10A and an antenna 10B, wherein the Doppler module 10A has characteristics similar to a Doppler radar. The antenna 10B receives a signal with a predetermined frequency, emits the signal to a living object such as a certain part of a human body which is continuously moving, and receives a reflected signal from the moving part. The reflected signal is sent to the Doppler module 10A. The frequency of the reflected signal is different from the original signal because of frequency drift, so the Doppler module 10A is capable of obtaining movement information of the certain part based on the frequency drift and the phase variation, between the original emitted signal and the received signal.

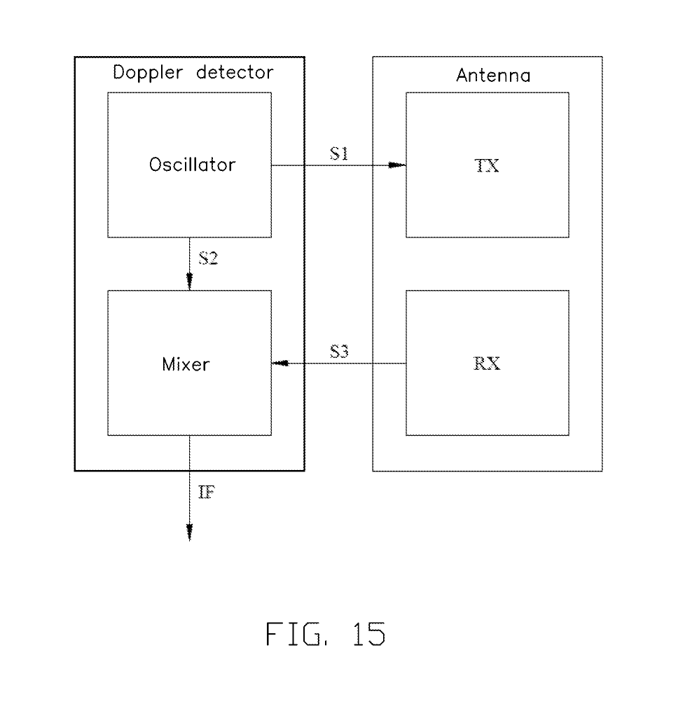

The Doppler detector is shown in FIG. 15. In one embodiment, the oscillator generates a signal at the frequency of 10.525 gigahertz (GHz). A signal Si is sent to the transmitting antenna Tx to emit electromagnetic wave. The emitted electromagnetic wave travels to an object under test and therefore reflected signal is received by the receiving antenna Rx. A signal S3 is demodulated and downconverted by the mixer MIX with a signal S2 from the oscillator OSC so that a baseband signal IF is generated.

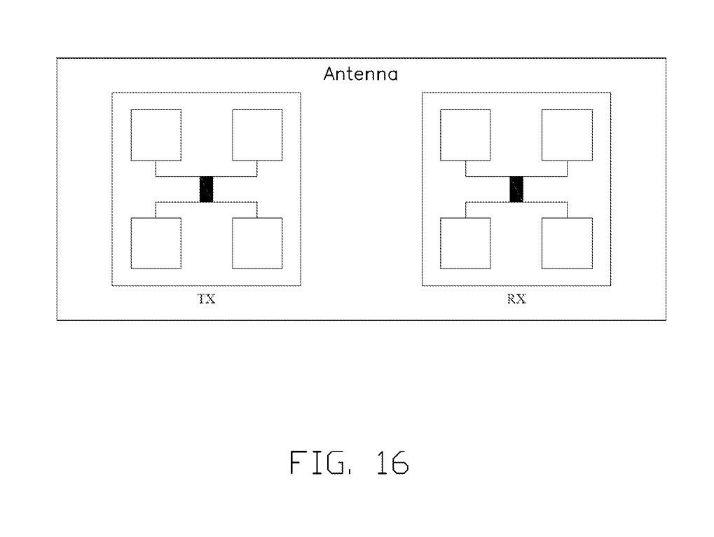

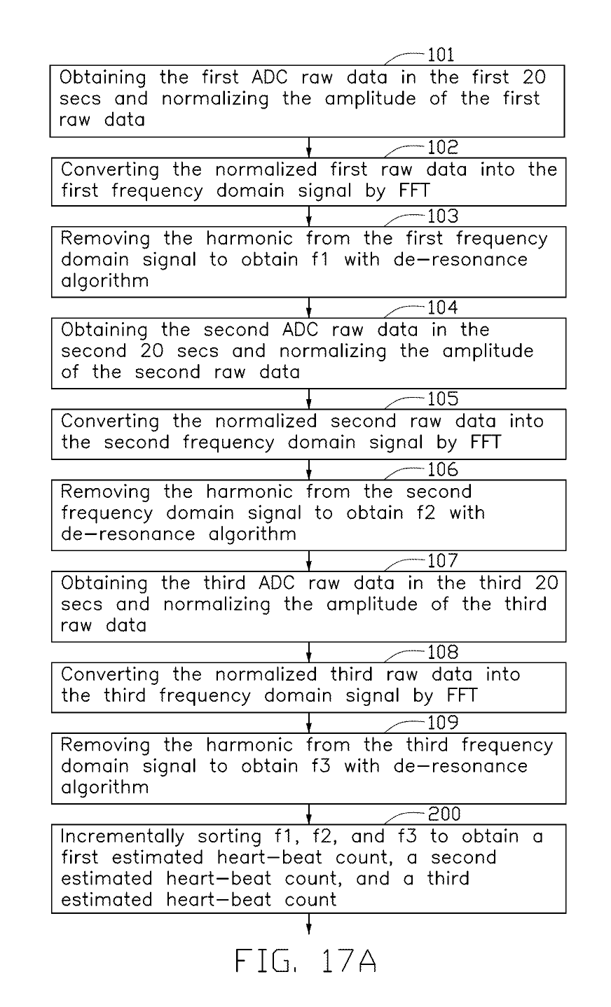

The antenna 10B includes terminals for emitting and receiving signals (not shown in the drawings). In one embodiment, these terminals are arranged in a 2-by-2 array and configured to emit and receive signals respectively, as shown in FIG. 16. The Doppler module 10A generates the emitted signal with the oscillator OSC and modulates the emitted signal, and demodulates and downconverts the received signal with the mixer so as to generate the baseband signal (IF) for outputting.

Preferably, the physiological signal sensor utilizes two Doppler detectors. One has the Doppler module only while the other one has an antenna in addition to the Doppler module.

The first amplification filter 20 and the second amplification filter 22 are essentially heart-beat counting analog circuit and respiration counting analog circuit.

As to the heart-beat analog circuit of the first amplification filter 20, the baseband signal from the first Doppler detector 10 has small amplitude, less than 10 millivolt (mV), and is amplified by the first amplification filter 20. The signal is then filtered by a filter so frequencies outside of the frequency of normal heart-beat (that is, outside the range from 0.72 Hz to 3.12 Hz) may be filtered out.

The filter may be the band pass filter with cutoff frequencies at 0.72 Hz and 3.12 Hz, that is, the pass band is from 0.72 Hz to 3.12 Hz. However, a single band pass filter may allow some signal with a frequency at the band boundary, and the response of such a band pass filter is not as good as a combination of a high pass filter and a low pass filter. Hence, the single band pass filter may be replaced by the combination of a high pass filter and a low pass filter. The order and/or the cutoff frequency of either the high pass filter or the low pass filter may be adjusted to that the frequency response thereof is sharper at the cutoff frequency and the off-band signals are better filtered. The heart-beat circuit has the high pass filter preceding the low pass filter. Hence, a high pass filter is used for filtering out the DC offset generated by the amplifier so that the signal is not saturated while being amplified. The high pass filter may amplify the signal twice and then the low pass filter can amplify the filtered-then-amplified signal twice. In another embodiment, there is an additional amplifier for further amplification.

As to the respiration analog circuit of the second amplification filter 22, the baseband signal from the first Doppler detector 10 has small amplitude, less than 10 millivolt (mV), and is amplified after being received by the first amplification filter 22. The signal is then filtered by the filter so the signal with out-of-range frequency (the frequency of normal respiration might be within the range from 0.066 Hz to 0.72 Hz) may be filtered out. Further, the filter may be a band pass filter with cutoff frequencies at 0.066 Hz and 0.72 Hz. Similarly, the band pass may not completely filter out the out-of-band signal. Hence, the single band pass filter may be replaced by the combination of a high pass filter and a low pass filter. The order and/or the cutoff frequency of either the high pass filter or the low pass filter may be adjusted to that the frequency response thereof is sharper at the cutoff frequency and the off-band signals are better filtered. The heart-beat circuit has the high pass filter preceding the low pass filter. Hence, a high pass filter is used for filtering out the DC offset generated by the amplifier so that the signal is saturated while being amplified. The high pass filter may amplify the signal twice and then the low pass filter can amplify the filtered-and-amplified signal by a factor of 1.5. In another embodiment, there is an additional amplifier for further amplification.

The analog signals from the heart-beat counting circuit and the respiration counting circuit are converted into digital signals by analog-to-digital converters (ADC) and then the digital signals are sent to the processor 30 for processing.

The first and second digital signals are essentially time-domain signals. The digital signal processing performed by the processor 30 includes a fast Fourier transform (FFT) for converting a time-domain signal into a frequency-domain signal, to obtain the fundamental frequency, and a de-resonance process so as to obtain the signal related to the respiration rate and the heart-beat rate.

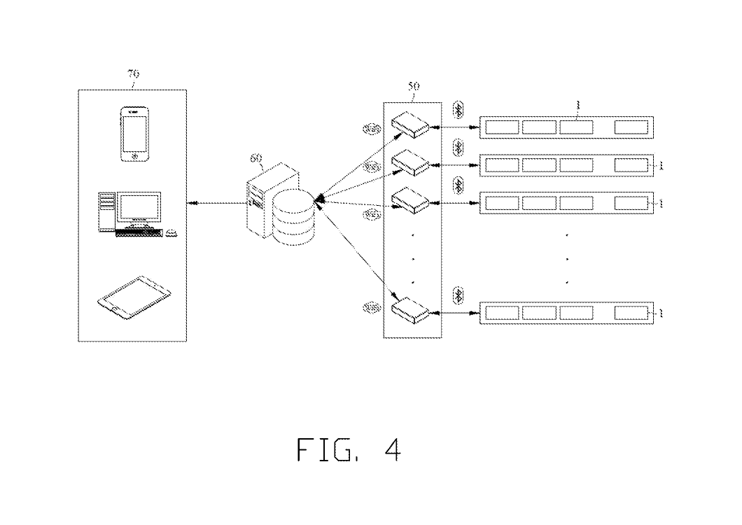

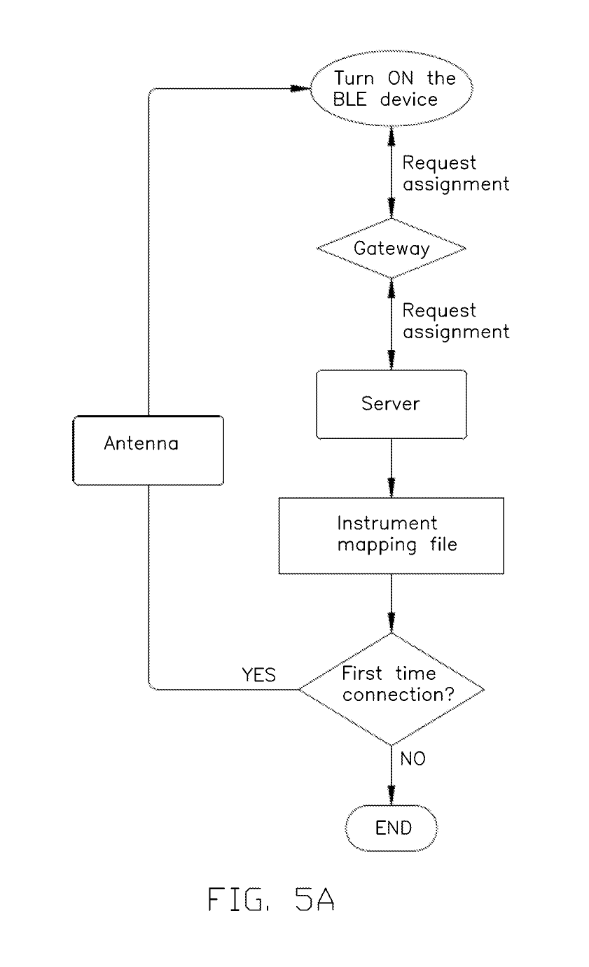

The transceiver 40 is preferably a wireless transceiver for portability. The transceiver 40 wirelessly transmits the heart-beat rate and the respiration rate via BLUETOOTH to the gateway 50, as shown in FIG. 4. The heart-beat rate and the respiration rate are then sent to the server 60 in the backend or displayed. Furthermore, the server 60 sends the corresponding physiological information to a remote view system (RVS) 70 for later processing and statistical and disease analysis.

Furthermore, the physiological signal sensor in the disclosure may further have a power management unit (not shown in drawings). The power management unit includes a battery for supplying electric power, an external power source for supplying electric power for charging battery, a charge circuit for charging the battery with the electric power, a power switch for turning device ON/OFF, a power management circuit for supplying varieties of electrical power to the device, an external power detector for detecting a status of the external power source, a processor for controlling the device and the power, and a status display for the device.

The power management circuit or unit allows the device to be turned off when not in use to save power. The device may therefore be turned off remotely by wireless. The device for displaying status may be shared and controlled by the processor to display the status of devices such as the power of battery, the charge status, the connection status, etc. The processor is capable of controlling the device for displaying status even if the device is powered off. The peripheral devices can be disabled or powered off when the device is in charging status. The processor may either keep the device constantly powered on or can turn off the device when an external input of the device is removed. The processor may detect the power of battery and prompt a warning of low battery. Further, when the battery is out of power or almost out of power, the processor may execute certain operation such as data storage or warning prompt before turning off the device so as to prevent loss.

The calculation of the respiration rate and the heart-beat rate is as follows.

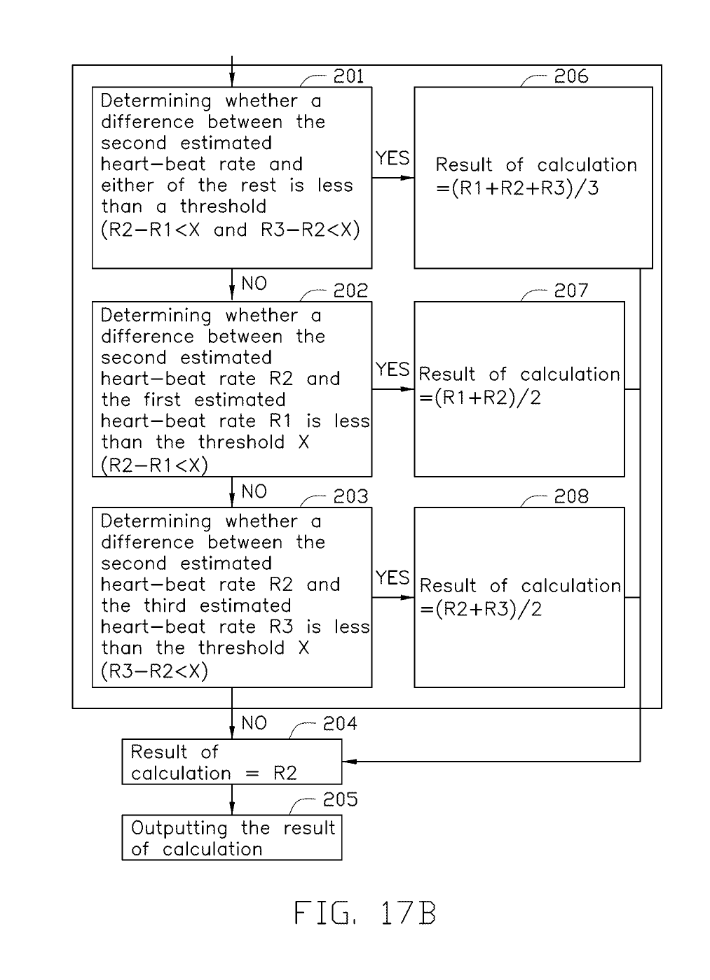

FIG. 17A and FIG. 17B together show a flow of the calculation process of the heart-beat rate in one embodiment of the disclosure. In this embodiment, three successive segments of raw data, each having a length of 20 seconds, are used for the estimation of the heart-beat rate. However, in another embodiment, three segments of raw data, each having a length of 10 seconds, in succession may be used for the calculation of the heart-beat rate. In yet another embodiment, the raw data of the sensed data corresponding to the first sixty seconds is used for the calculation of the first heart-beat rate while the raw data of the sensed data corresponding to the period from 21^{st} second to 80^{th} second is used for the calculation of the second heart-beat rate.

The calculation of heart-beat rate includes the following steps.

Step 101: the processor obtains the first raw data in the first twenty seconds and normalizes the amplitude of the first raw data.

The reason for the need of normalization of the amplitude is that the physical condition of the user and the manner of use may result in variation of the amplitude of the signal received by the sensor. After the normalization, the amplitude of the signal is normalized to a specific range and the influence from the manner of use is reduced. Further, the DC component in the signal needs to be removed in normalization because the result of FFT would provide a peak at DC if a DC component exists in the signal.

Step 102: fast Fourier transform (FFT) is performed for converting the normalized first raw data into a first frequency domain signal.

Step 103: the first frequency domain signal is processed with a de-resonance algorithm to remove any harmonics to obtain a first frequency f₁ which is the fundamental frequency of the first frequency domain signal.

Step 104: the second raw data in 21^{st} second to 40^{th} second is obtained and normalized in amplitude.

Step 105: the normalized second raw data is converted into a second frequency domain signal by FFT.

Step 106: the second frequency domain signal is processed with the de-resonance algorithm to remove harmonics to obtain a second frequency f₂ which is the fundamental frequency of the second frequency domain signal.

Step 107: the third raw data in 41^{st} second to 60^{th} second is obtained and normalized in amplitude.

Step 108: the normalized third raw data is converted into a third frequency domain signal by FFT.

Step 109: the third frequency domain signal is processed with the de-resonance algorithm to remove harmonics to obtain a third frequency f₃ which is the fundamental frequency of the third frequency domain signal.

Step 200: the first frequency f₁, the second frequency f₂, and the third frequency f₃ are sorted incrementally so that a first estimated heart-beat rate R1, a second estimated heart-beat rate R2, and a third estimated heart-beat rate R3 are obtained. The first estimated heart-beat rate has the least value whilst the third estimated heart-beat rate for the greatest value.

Step 201, whether any difference between the second estimated heart-beat rate R2 and either of the others is less than a threshold X is then determined. Explicitly, whether the value of the second estimated heart-beat rate R2 minus the first estimated heart-beat rate R1 is less than the threshold or whether the value of the third estimated heart-beat rate R3 minus the second estimated heart-beat rate R2 is less than the threshold X is determined. Herein, the threshold X is an error of tolerance and may be set as five in one embodiment. If the determination in the step 201 is negative or false, the step 202 is executed; otherwise, the step 206 is executed.

Step 202: whether a difference between the second estimated heart-beat rate R2 and the first estimated heart-beat rate R1 is less than the threshold X is determined. If the determination in the step 202 is negative or false, the step 203 is executed; otherwise, the step 207 is applied.

Step 203: whether a difference between the second estimated heart-beat rate R2 and the third estimated heart-beat rate R3 is less than the threshold X is determined. If the determination in the step 203 is negative or false, the step 204 is applied, otherwise the step 208 is applied.

Step 204: the median of the three estimated heart-beat rates, i.e. in this case the second estimated heart-beat rate R2, is selected as the heart-beat rate.

Step 205: the result of calculation, the heart-beatrate, is outputted.

Step 206: an average of the three estimated heart-beat rates, which is one third the sum of the first estimated heart-beat rate R1, the second estimated heart-beat rate R2, and the third estimated heart-beat rate R3, is determined to be the result of calculation of the heart-beat rate.

Step 207: an average of the first estimated heart-beat rate R1 and the second estimated heart-beat rate R2, i.e. half the sum of the first estimated heart-beat rate R1 and the second estimated heart-beat rate R2, is determined as being the result of calculation of the heart-beat rate.

Step 208: an average of the second estimated heart-beat rate R2 and the third estimated heart-beat rate R3, i.e. half the sum of the second estimated heart-beat rate R2 and the third estimated heart-beat rate R3, is determined upon as being the result of calculation of the heart-beat rate.

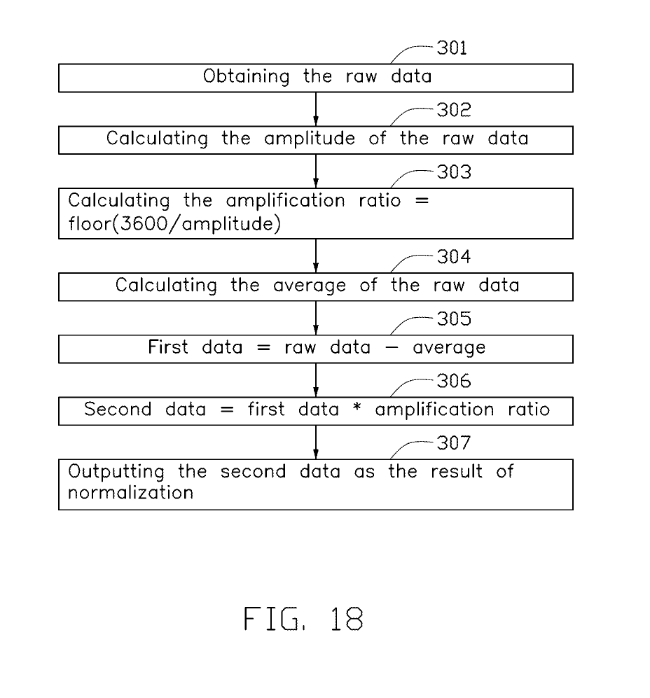

FIG. 18 shows a flowchart of the amplitude normalization in one embodiment of the disclosure. The amplitude normalization includes the steps of Step 301, the processor obtains a raw data from the sensor. Step 302, an amplitude of the raw data is calculated. Step 303, an amplification ratio defined as the integer part of the result of 3600 divided by the amplitude of the raw data is calculated. Step 304, an average of the raw data is calculated. Step 305, a first data is obtained by subtracting the average from each piece of raw data. Step 306, a second data is obtained by multiplying the first data by the amplification ratio. Then by step 307, it is the second data, the normalized raw data, which is outputted.

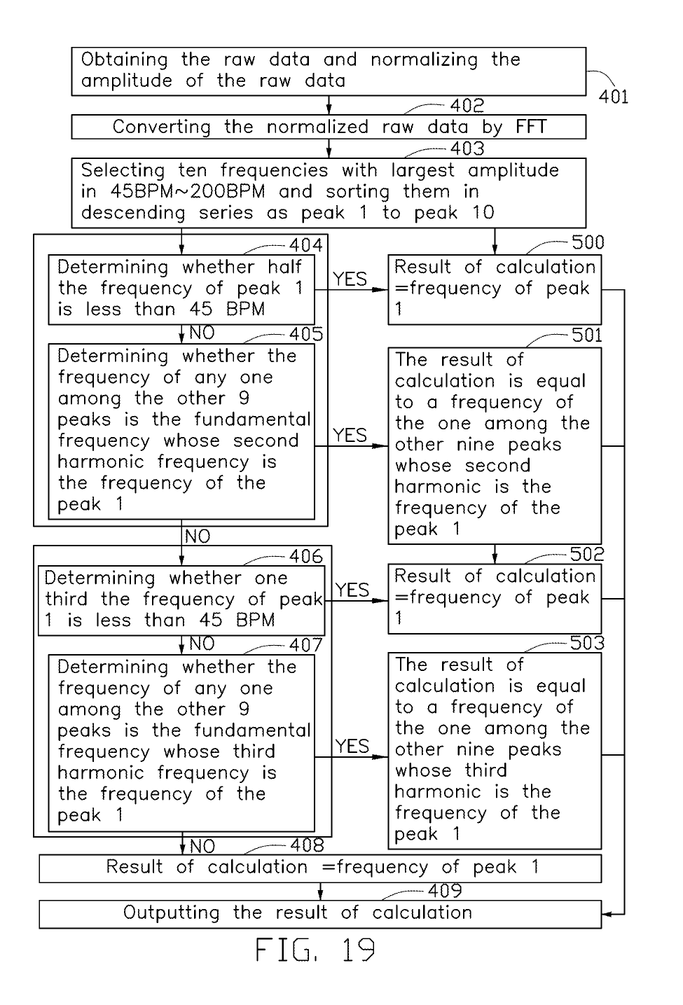

FIG. 19 shows a flowchart of a de-resonance algorithm in one embodiment of the disclosure, the flow includes the following steps.

Step 401: the processor obtains the raw data and normalizes the amplitude of the raw data.

Step 402: the normalized raw data is converted by FFT.

Step 403: within the range from 45 BPM to 200 BPM, ten frequencies which have amplitudes larger than other frequencies are selected and sorted in a descending series of amplitude, as peak 1 to peak 10.

Step 404: whether half the frequency of the peak 1 is less than 45 BPM is determined. The step 405 is applied if determination is affirmative, the step 500 is otherwise applied. The result of calculation is given as the frequency of the peak 1 in the step 500.

Step 405: whether the frequency of any one among the other 9 peaks is the fundamental frequency having second harmonic frequency equal to peak 1 frequency is determined. In this determination, two conditions are considered (first), whether half the frequency of the peak 1 is substantially equal to a frequency among the other 9 frequencies, i.e. whether the difference therebetween is less than a certain threshold. (Second), whether the ratio between the amplitude of the peak in comparison and the amplitude of peak 1 is larger than a certain value, e.g. 50%.

If the result of determination in the step 405 is negative, the step 406 is applied, the step 501 is chosen otherwise. In the step 501, the result of calculation is equal to a frequency of the one, or the first one, among the other nine peaks which have second harmonic at the frequency of the peak 1.

Step 406: whether one third of the frequency of the peak 1 is less than 45 BPM is determined. If the result of determination is negative, the step 407 is applied, the step 502 is applied otherwise. In the step 502, the result of calculation is given as the frequency of the peak 1.

Step 407: a determination is made as to whether the frequency of any one among the other 9 peaks is the fundamental frequency which has third harmonic frequency as the frequency of the peak 1. Both of the following conditions must be met:

One: whether one third of the frequency of the peak 1 is substantially equal to a frequency among the frequencies of the other 9 peaks, i.e. the difference therebetween being less than a certain threshold is seen as substantially equal and

Two: the ratio between the amplitude of the peak being compared and the amplitude of peak 1 being larger than a certain value, e.g. 50%.

If the result of determination in step 407 is negative, the step 408 is applied, the step 503 being applied otherwise. In the step 503, the result of calculation is equal to a frequency of the one, or the first one, among the other nine peaks which has third harmonic equal to the frequency of the peak 1.

Step 408: the result of calculation is given as the frequency of peak 1.

Step 409: the result of calculation is outputted.

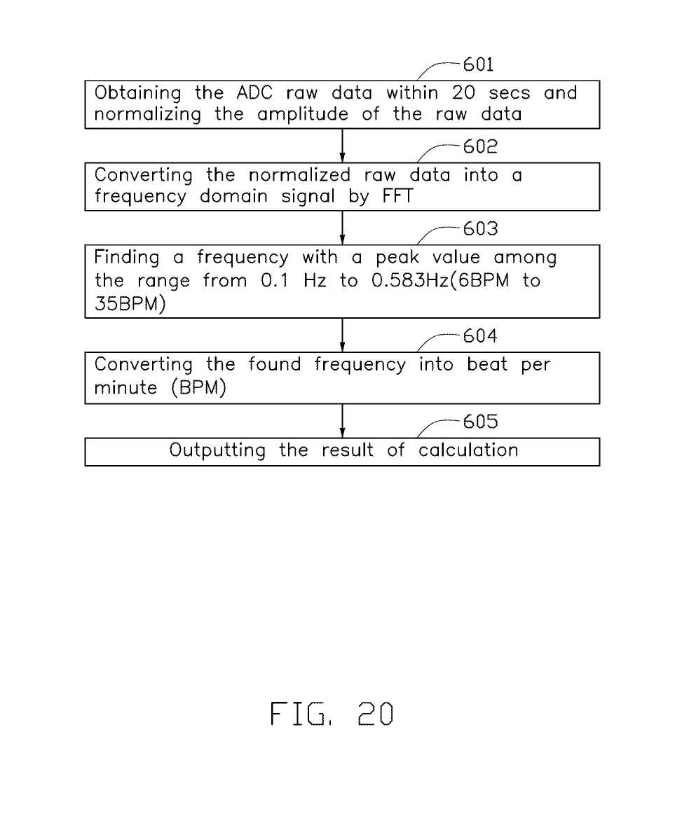

FIG. 20 shows an algorithm for calculating the respiration rate in one embodiment of the disclosure. The flow includes the following steps.

Step 601: a raw data of a period of 20 seconds is obtained and normalized in amplitude.

Step 602: the normalized raw data is converted into the frequency domain by FFT.

Step 603: a frequency with a peak value among the range from 0.1 Hz to 0.583 Hz is found.

Step 604: the found frequency is converted into breathes per minute (BPM) as a result of calculation of the respiration rate.

Step 605: the result of calculation is outputted.

Step 601 and the step 602 are possibly accomplished during the estimation of the heart-beat rate, so the processor may directly execute the step 603 after the result in the step 602 is obtained.

Furthermore, the physiological signal sensor in the disclosure is preferably worn on a certain location such as above the clavicle for measuring the respiration. The muscles and ribs around the chest move in respiration, and thus the physiological signal sensor may accordingly obtain the respiration rate. Further, the physiological signal sensor may be worn above the artery around the neck for measuring the heart-beat rate. The artery periodically pulses as the heart beats, so the physiological signal sensor may accordingly obtain the heart-beat rate.

Hence, the physiological signal sensor may be disposed on a certain location on the user's body and a plurality of sensors may be connected together. For example, the physiological information such as respiration rate and/or heart-beat rate may be detected if the physiological signal sensor is disposed near the clavicle or artery.

As to the appearance of the physiological signal sensor in the disclosure, the sensor is supposed to be put near the neck artery and the clavicle, so the appearance when the physiological signal sensor is worn is taken into consideration. In one embodiment, the physiological signal sensor resembles a necklace. For example, the necklace-like physiological signal sensor may be fastened around the neck so that the measurement is not affected even when the location of the sensor varies.

In the disclosure, the physiological information such as respiration rate and the heart-beat rate obtained by the physiological signal sensor is sent via BLUTOOTH module (BLE) to the gateway, then received by the server via the WI-FI module of the gateway, and saved in the corresponding fields in the database. The remote view system (RVS) provides a variety of interfaces, such as cell phone apps, personal computer, and the tablet, for displaying related physiological information such as respiration rate and the heart-beat rate. Each gateway is capable of being connected to a plurality of physiological signal sensors for transferring the data therefrom to a backend server for processing.

As to how the physiological signal sensor is connected to the server, when the physiological signal sensor is connected to the server for the first time, the clock in the physiological signal sensor is corrected by the server based on a network time protocol (NTP). Afterwards, the physiological signal sensor collects and sends the data sequentially via the gateway to the server for saving the data in the corresponding database field.

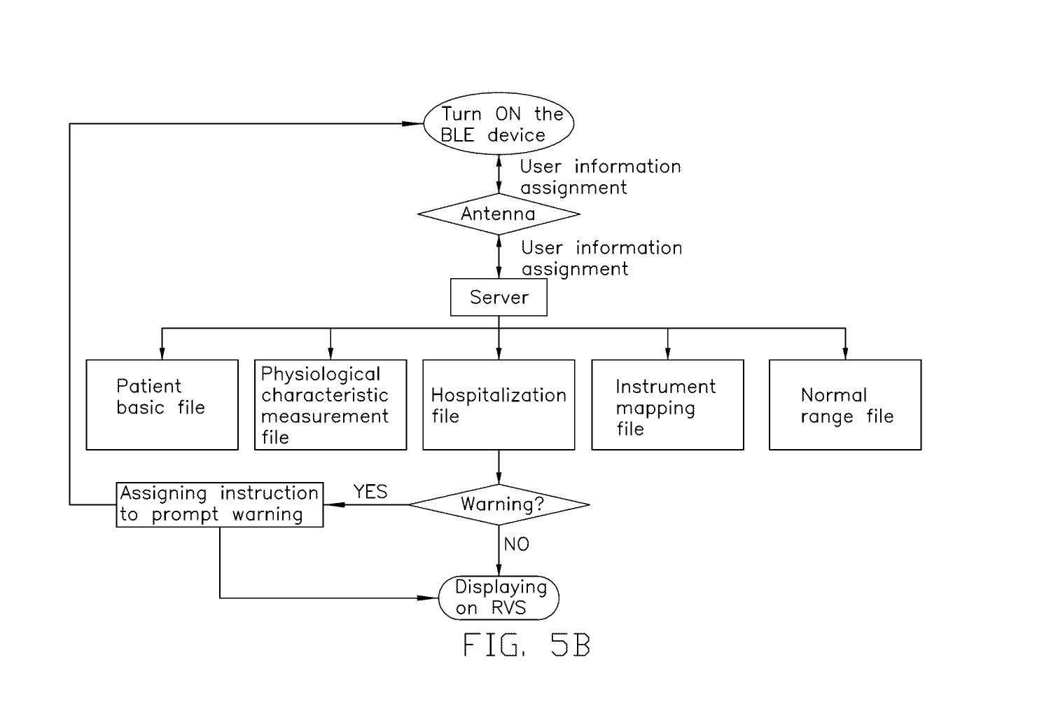

FIG. 5A and FIG. 5B show the flows of process in the embodiment about data flow from the sensor to the server and the data access.

In FIG. 5A, the data flow from physiological signal sensor to the server includes the step in which the physiological signal sensor is connected to the server for the first time. The clock of the physiological signal sensor is corrected by the server based on the network time protocol (NTP) to synchronize the clock of the physiological signal sensor. Afterwards, the physiological signal sensor collects and sends the data sequentially via the gateway to the server for saving the data in the corresponding database field.

In FIG. 5B, the data access flow includes building data tables and the fields therein in the database. Hence, the server may directly get the corresponding field by enabling data service when the data in received by the server. Furthermore, the server can determine whether the received physiological readings are within the reasonable range. If the readings exceed the reasonable range, the server can send a warning to the sensor and the remote view system (RVS) so as to inform the user and the department in charge.

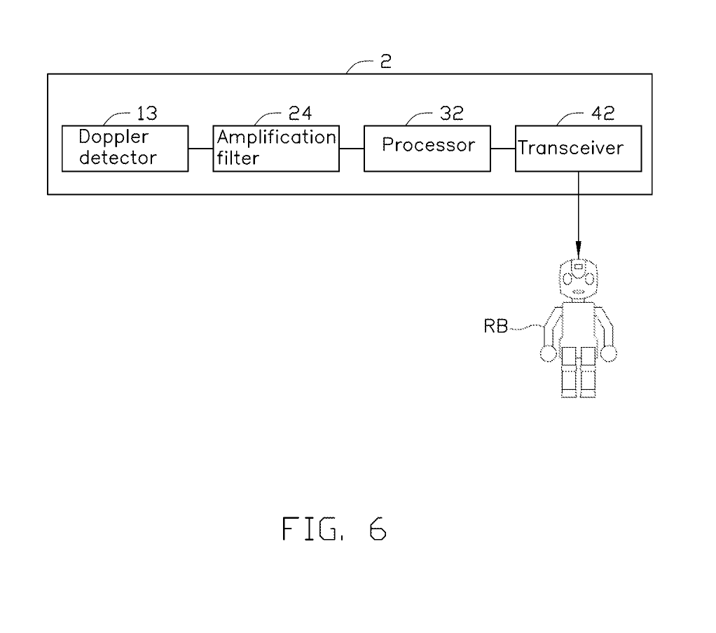

FIG. 6 shows how the physiological signal sensor interacts with a robot. In the embodiment, the physiological signal sensor 2 is configured to detect and process the gesture of a user and then send it to the robot so that the robot may act according to the gesture. For example, the robot can move to the user when it detects a beckoning by the user. When the robot detects the user waving goodbye, the robot can wave back. The user may arbitrarily set the robot to do different actions in response to different gestures.

The Doppler detector 13 sends an RF signal and receives a reflected RF signal to generate a baseband signal. The baseband signal is filtered by the amplification filter 24 and then the component of the baseband signal with frequency ranging from zero to 40 Hz is sent to the processor 32. The processor 32 processes the received signal with certain procedures such as Fourier transform and then sends the processed signal to the transceiver 42 for sending to the robot RB. In another embodiment, the amplification filter 24 may directly send the filtered signal to the robot RB for processing if the hardware performance of the robot RB is better than that of the processor 32.

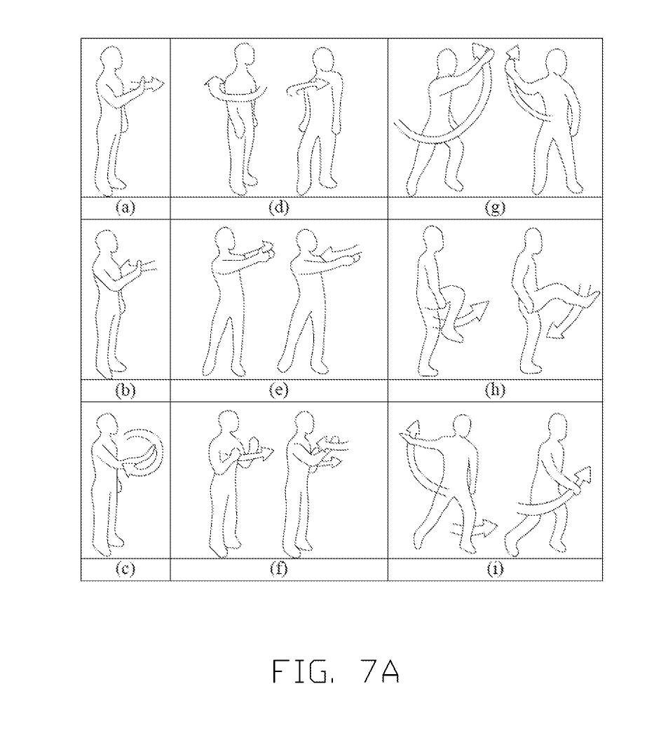

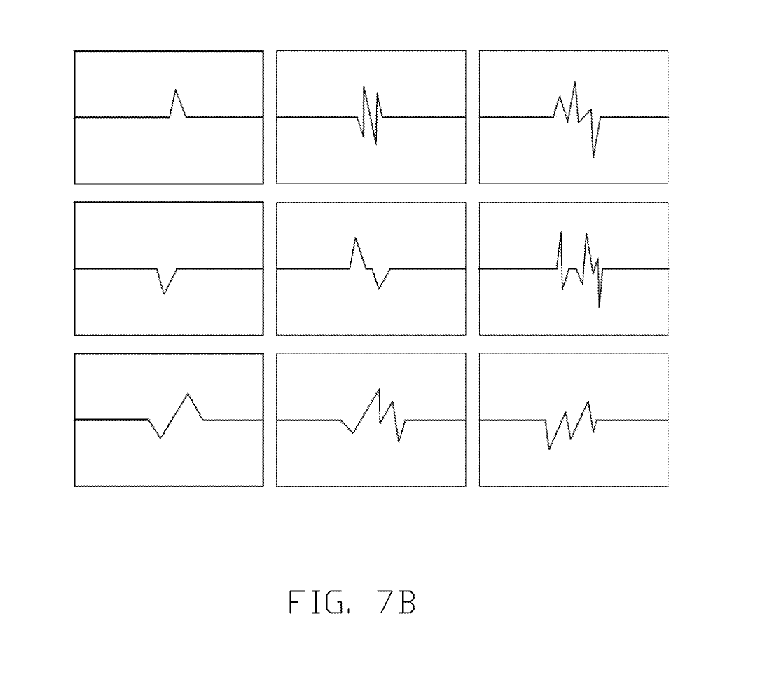

As shown in FIG. 7A, gestures are categorized into a plurality of categories such as pushing hand, pulling hand, swinging hand, swinging body, hand up, oblique extension, bending, or kicking, raising leg, swinging leg, and/or a combination. FIG. 7A and FIG. 7B are not to limit the scope of the disclosure in this respect. When the Doppler detector detects the signal, the Doppler detector converts the signal into a frequency-time signal as shown in FIG. 7B. Different gestures correspond to different frequency-time signals, so the robot is capable of recognizing the gesture based on the frequency-time signal.

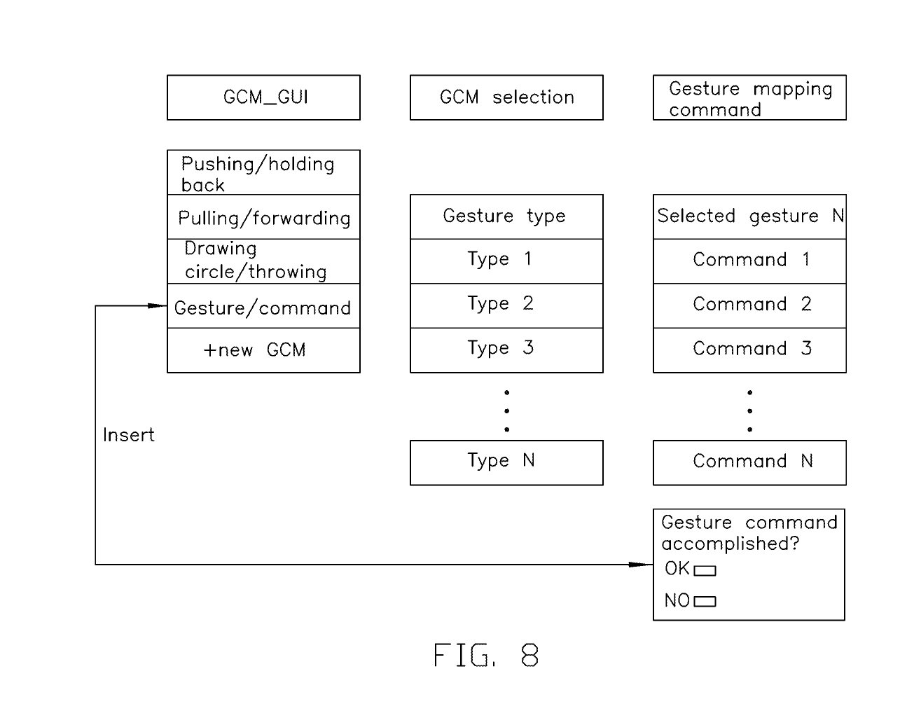

As shown in FIG. 8, the setting of the gesture command (GCM) may be done through the gesture and command mapping graphical user interface (GCM_GUI). Further, the GCM_GUI also provides the functionality of accepting new commands.

Another gesture recognition method in the disclosure is to capture video streams by the CCD camera of the time-of-flight (TOF) camera on the head of the robot RB.

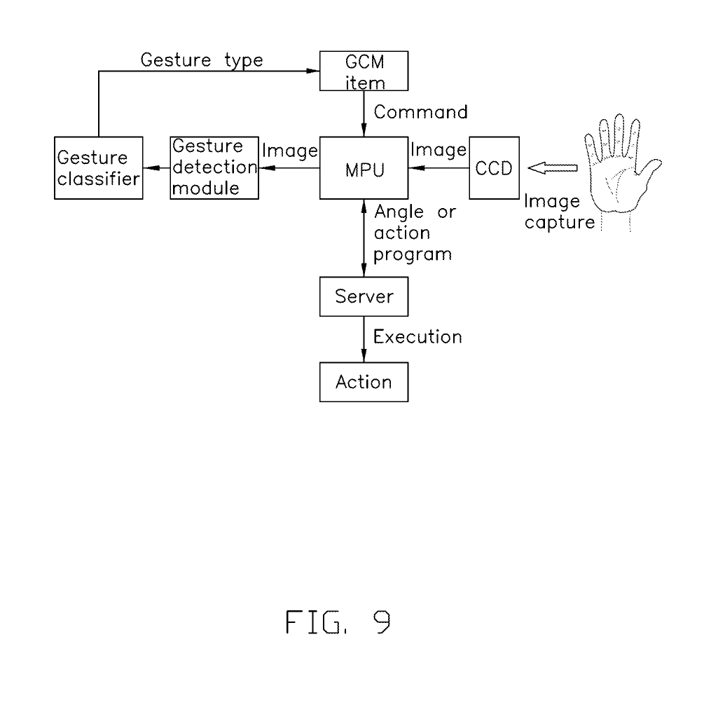

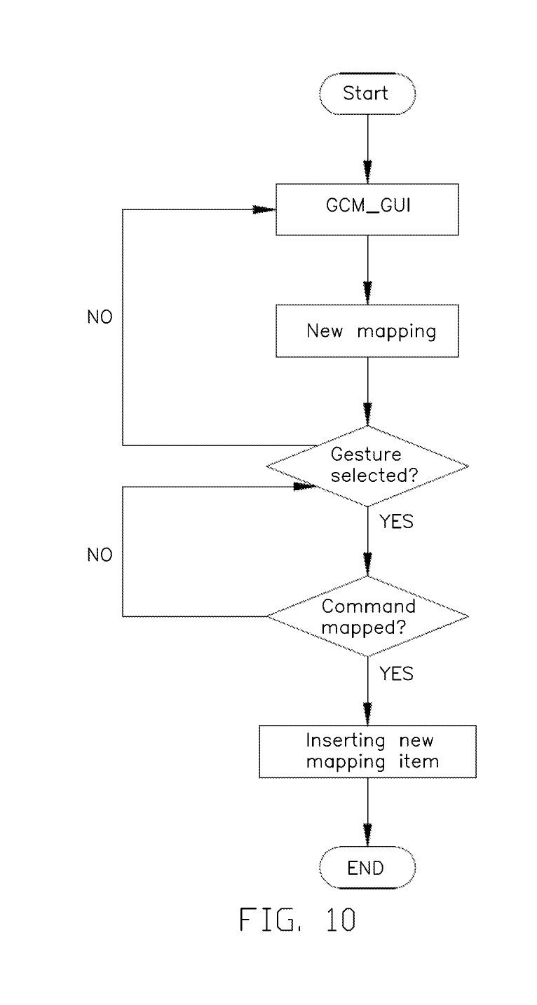

Thereby, gestures in the video stream are obtained by the gesture recognition sensor, and corresponding commands after the processor 32 determines the classification of the gesture are generated so that the robot is RB is capable of executing the required action. As shown in FIG. 9, what is utilized in hardware includes microprocessor unit (MPU), charge-coupled device (CCD) camera, time-of-flight (TOF) camera, light filter, and color filter. What is utilized in software includes:

1. camera calibration.

2. Morphology method.

3. Region of Interest (ROI).

4. Convolution filter.

5. Convolution contours enhancement.

6. Convexity Defects.

7. Convex Hull.

8. Random transformation.

9. Hough transformation.

10. Background image subtraction.

11. Color filtering.

12. Optical flow

13. Depth imaging.

14. Gestures classification.

15. Hidden Markov Models.

16. Dynamic Time Warping.

17. Machine learning methods.

18. Support Vector Machines.

19. K-nearest neighbors.

20. Gestures database.

21. Gesture and command mapping GUI editing.

Explicitly, the microprocessor unit (MPU) first calibrates the charge-coupled device (CCD) camera, such as geometric correction, aberration, or camera model, for the operation and the accuracy of the calculation. Such calibration is, in one embodiment, performed before the robot is purchased and the input parameters may be saved. In another embodiment, the calibration is performed before the following flow processes.

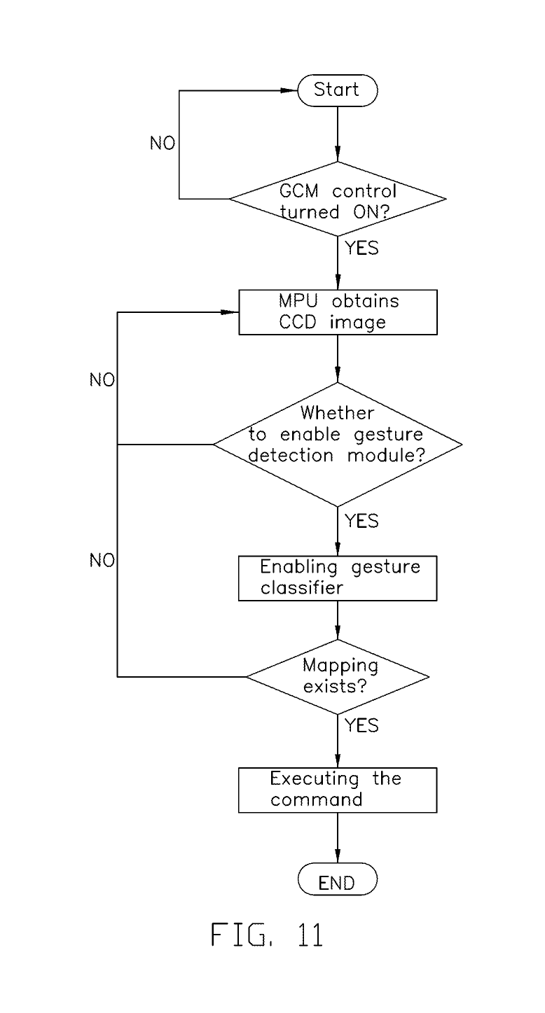

As to setup, as shown in FIG. 10, the setup operation includes steps of start, enter GCM_GUI, perform new mapping, determine whether a gesture is selected, determine whether a command is mapped, insert new mapping item, and end.

The MPU reads raw data of a series of images from the CCD camera. The MPU processes the raw data with image processing methods such as Morphology method, Region of Interest, Convolution filter, Convolution contours enhancement, and others so as to obtain processed data of the images. The processed image data has better quality in sharpness, contrast, edge, and serration ratio than the raw data. At least one self-defined feature may be obtained from the processed data by convexity defects, convex hull, random transform, Hough transform, background image subtraction, color filter, optical flow, and/or depth of image. The feature is classified by the gestures classifier so as to obtain a gesture database. The gestures classifier classifies the gestures by means such as dynamic time warping, hidden Markov models, K-nearest neighbors, support vector machines, and others. Each gesture in the gesture database may be set so as to correspond to a gesture command via the gesture and command mapping GUI editor.

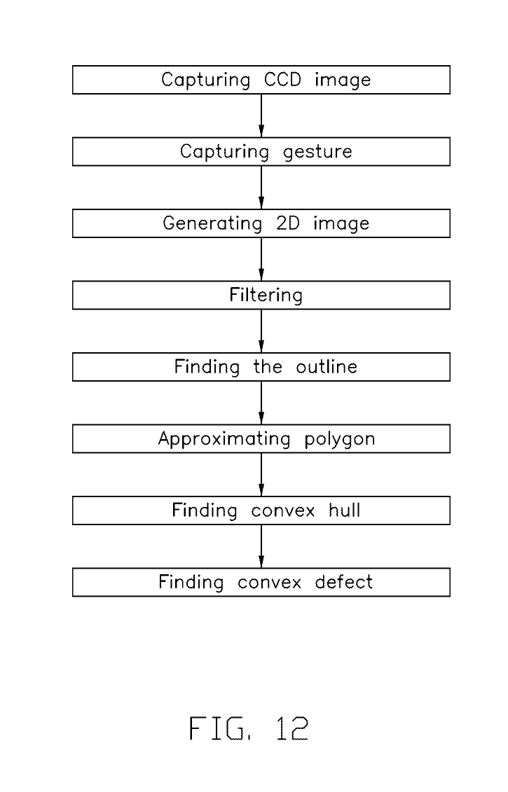

As shown in FIG. 11, the operation flow includes the steps of start, determining whether the GCM control is turned ON, the MPU obtaining the CCD image, determining whether to enable the gesture detection module, enabling the gesture classifier, determining whether a mapping exists, executing a command, and end. Hence, the MPU takes the gesture type into the gesture database so as to fix the gesture command correspondingly. The robot is able to perform corresponding actions.

FIG. 12 shows an exemplary example of the pipeline algorithm of the gesture detection module in second embodiment of the disclosure. The algorithm includes capturing a CCD image, capturing a gesture by optical flow, sub-imaging, generating 2D image, filtering such as by convolution filter, establishing outline such as watershed, serpentine curve, and the like, approximating polygon, finding convex hull, and finding convexity defect.

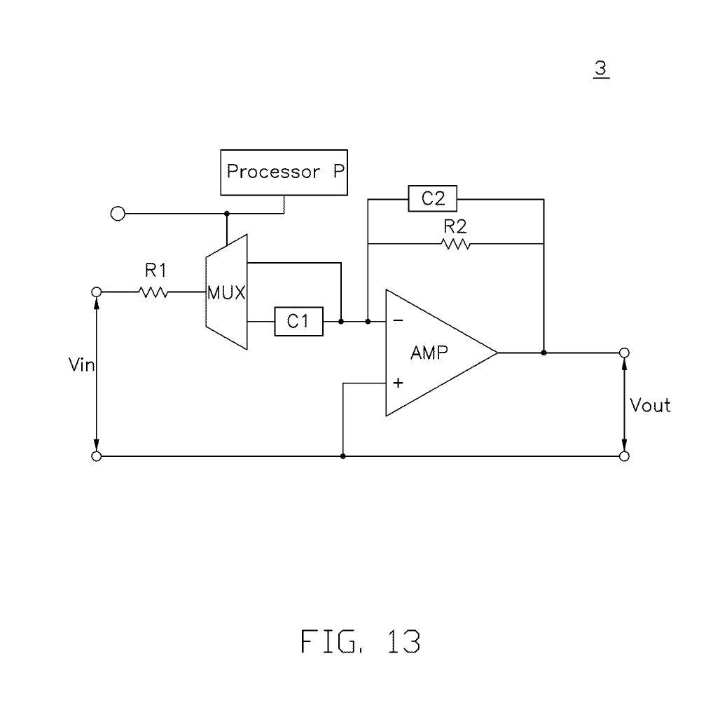

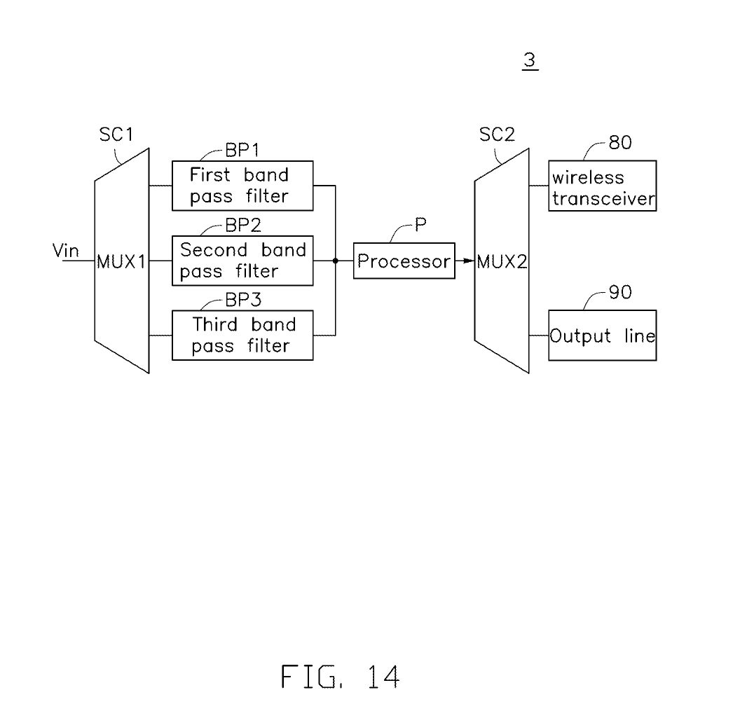

FIG. 13 shows a band pass filter in a physiological signal sensor in one embodiment of the disclosure. The band pass filter is controlled by the processor P of the physiological signal sensor and is capable of dynamically adjusting the pass band of the band pass filter. In the embodiment, the signal defined between the input terminals is the baseband signal IF and the output signal Vout is sent to the processor P for processing.

The gain and the frequency responses of the band pass filter are shown as:

Gain G=−R1/R2.

fcL=½πR1C1.

fcH=½πR2C2.

The pass band of the band pass filter is defined by the fcH and the fcL.

One terminal of the resistor R1 is connected to a positive input terminal of the physiological signal sensor 3 for receiving a baseband signal while the other terminal of the resistor R1 is connected to an input terminal of the multiplexer MUX. The multiplexer MUX is controlled by a trigger signal for selecting a path for outputting the baseband signal. The trigger signal is also sent to the processor P so that the processor P is capable of adjusting the capacitance of the adjustable capacitor C1 and/or the capacitance of the adjustable capacitor C2 for changing the pass band of the band pass filter.

In the disclosure, the heart-beat rate is estimated based on the signal with a frequency within the range from 0.72 Hz to 3.12 Hz. The respiration rate is estimated based on the signal with frequency within the range from 0.066 Hz to 0.72 Hz, and the gesture recognition is performed for the signal with frequency within the range from zero (DC) to 40 Hz.

The multiplexer MUX is controlled by external trigger signal for selecting conduction path. When the physiological signal sensor is coupled to the robot, the external signal or the trigger signal makes the multiplexer MUX select the path corresponding to the logic value 1 (true) and the cutoff frequency of the band pass filter is DC. After the processor P receives the trigger signal, the processor P adjusts the capacitance of the adjustable capacitor C2 so as to adjust the pass band of the band pass filter into the range from DC to 40 Hz.

The external signal or the trigger signal may be generated in several ways. For example, the physiological signal sensor has a near field communication (NFC) module and the robot has an NFC module as well. After the NFC module in the physiological signal sensor receives and verifies the signal from the NFC module in the robot, the NFC module in the physiological signal sensor generates an external interrupt signal or the trigger signal to the multiplexer. Hence, the input signal Vin is not coupled via the first adjustable capacitor C1. Meanwhile, the controller in the physiological signal sensor adjusts the capacitance of the second adjustable capacitor C2 so as to bring the pass band of the band pass filter within DC to 40 Hz.

In another embodiment, the physiological signal sensor has a female connector and the robot has a corresponding male connector, so a pin of the female connector generates the trigger signal while the physiological signal sensor is connected to the robot to control the multiplexer to switch the path. The controller in the physiological signal sensor controls the capacitance of the second adjustable capacitor C2 so that the pass band of the band pass filter is within the range of DC to 40 Hz.

In yet another embodiment, the physiological signal sensor has a male connector and the robot has a corresponding female connector. When the physiological signal sensor is connected to the robot, a trigger signal is generated from a certain pin of the male connector so as to control the multiplexer to switch the path. The controller in the physiological signal sensor controls the capacitance of the second adjustable capacitor C2 so that the pass band of the band pass filter is brought within the range from DC to 40 Hz.

When the physiological signal sensor operates in the physiological signal measuring mode, the multiplexer MUX is switched to the path corresponding to the value 0. Meanwhile, the processor P adjusts the capacitance of the first adjustable capacitor Cl and/or the capacitance of the second adjustable capacitor C2 based on whether the measured physiological signal is heart-beat rate or respiration rate, so as to change the pass band of the band pass filter.

FIG. 14 shows a band pass filter in a physiological signal sensor in one embodiment of the disclosure. In this embodiment, the band pass filter includes a first band pass filter BP1, a second band pass filter BP2, and a third band pass filter BP3. By the switching of the first multiplexer MUX1, the processor P receives the correct filtered baseband signal. The first multiplexer MUX1 is controlled by the first selection signal SC1 generated by the processor P. The first band pass filter BP1 allows the signal with frequency within the range from 0.72 Hz to 3.12 Hz to pass, and the second band pass filter BP2 allows the signal with frequency within the range from 0.066 Hz to 0.72 Hz to pass. The third band pass filter BP3 allows the signal with frequency within the range from DC to 40 Hz to pass. Furthermore, the physiological signal sensor 3 determines whether the physiological signal sensor is connected to the robot based on a specific mechanism such as wireless detection utilized in near field communication protocol or via the physical connector.

The signal filtered by the band pass filter is, for example, amplified again by the amplifier (not shown), and then the filtered-and-amplified signal is sent to the processor P for fast Fourier transform (FFT). The signal is transformed into frequency domain and the heart-beat rate and the respiration are estimated therefrom.

In another embodiment, the physiological signal sensor, in the gesture recognition mode, performs either a short-time Fourier transform, a wavelet transform, or a Hilbert-Huang transform on the filtered signal so as to obtain a time-frequency spectrum of the signal for gesture recognition. The information generated by the physiological signal sensor is sent to the robot for gesture recognition. The physiological signal sensor being either wirelessly or by-wires connected to the robot, the multiplexer MUX2 selects a correct path for the processor P sending the information to the robot, so that the robot performs the action based on the correct gesture recognition.

For example, if the physiological signal sensor is wirelessly connected to the robot, the selection signal SC2 makes the multiplexer MUX2 send the signal to the wireless transceiver 80 of the physiological signal sensor so that the wireless transceiver 80 sends the gesture recognition signal to the robot. If the physiological signal sensor is connected to the robot via a connector, the selection signal SC2 makes the multiplexer MUX2 send the signal to the connector of the physiological signal sensor so that the information in the signal is sent to the robot via the output line. The aforementioned output line is not limited to being a connecting wire only, but can be circuitry on printed circuit board (PCB).

What is claimed is:

1. A physiological signal sensor operable in a physiological signal measuring mode and a gesture recognition mode, the physiological signal sensor comprising:
a Doppler detector configured to emit a first RF signal with a predetermined frequency, to receive a second RF signal which is a reflected first RF signal, and to generate a baseband signal based on the first RF signal and the second RF signal;
a first processor configured to generate a detection result based on the baseband signal; and
a wireless transceiver configured to send the detection result to a server;
wherein the detection result comprises a heart-beat rate and a respiration rate when the physiological signal sensor operates in the physiological signal measuring mode, and the detection result is sent to an electronic device for a gesture recognition when the physiological signal sensor operates in the gesture recognition mode.

2. The physiological signal sensor in claim 1, further comprising a detection device configured to detect whether the physiological signal sensor is electronically connected to a robot, if the physiological signal sensor is electronically connected to the robot, the detection device generates a trigger signal to inform the first processor so that the physiological signal sensor operates in the gesture recognition mode.

3. The physiological signal sensor in claim 2, wherein the detection device is one of a near field communication (NFC) module and a connector capable of being connected to the robot.

4. The physiological signal sensor in claim 1, further comprising a band pass filter coupled to the Doppler detector, wherein a pass band of the band pass filter is determined based on an operation mode of the physiological signal sensor.

5. The physiological signal sensor in claim 4, wherein if the operation mode of the physiological signal sensor is the gesture recognition mode, the pass band of the band pass filter ranges from DC to 40 Hz.

6. The physiological signal sensor in claim 4, wherein if the operation mode of the physiological signal sensor is the physiological signal measuring mode and the first processor measures the heart-beat rate, the pass band of the band pass filter ranges from 0.72 Hz to 3.12 Hz.

7. The physiological signal sensor in claim 4, wherein if the operation mode of the physiological signal sensor is the physiological signal measuring mode and the first processor measures the respiration rate, the pass band of the band pass filter ranges from 0.066 Hz to 0.72 Hz.

8. The physiological signal sensor in claim 4, wherein the band pass filter determines the operation mode of the physiological signal sensor based on a trigger signal generated if the physiological signal sensor is connected to a robot.

9. The physiological signal sensor in claim 8, further comprising a connector, wherein when the connector is connected to the robot, the trigger signal is generated on one pin of the connector.

10. The physiological signal sensor in claim 8, wherein the band pass filter comprises:
an amplifier having a positive input terminal, a negative input terminal and an output terminal, wherein the positive input terminal is coupled to the Doppler detector;
a first resistor having one terminal coupled to the Doppler detector;
a first adjustable capacitor having one terminal coupled to the negative terminal, wherein the first adjustable capacitor is coupled to the first resistor in series;
a second resistor having one terminal coupled to the negative input terminal and the other one terminal coupled to the output terminal;
a second adjustable capacitor coupled to the second resistor in parallel; and
a second processor configured to adjust a capacitance of the first adjustable capacitor and/or a capacitance of the second adjustable capacitor based on the trigger signal.

11. The physiological signal sensor in claim 10, wherein the band pass filter further comprises a multiplexer comprising:
a multiplexer input terminal coupled to the first resistor;
a first multiplexer output terminal coupled to the capacitor;
a second multiplexer output terminal directly connected to the negative input terminal of the amplifier; and
a selecting terminal coupled to the second processor.

12. The physiological signal sensor in claim 4, wherein the band pass filter comprises:
a low pass filter; and
a high pass filter precedent to the low pass filter;
wherein the low pass filter is coupled to the Doppler detector via the high pass filter.

13. The physiological signal sensor in claim 1, wherein a shape of the physiological signal sensor is a necklace.

14. The physiological signal sensor in claim 1, wherein the processor obtains a first estimated heart-beat rate, a second estimated heart-beat rate, and a third estimated heart-beat rate from the baseband signal, and the processor determines the heart-beat rate based on a relationship between the first estimated heart-beat rate, the second estimated heart-beat rate, and the third estimated heart-beat rate.

15. The physiological signal sensor in claim 14, wherein the third estimated heart-beat rate is larger than the second estimated heart-beat rate, and the second estimated heart-beat rate is larger than the first estimated heart-beat rate.

16. The physiological signal sensor in claim 15, wherein if a difference between the second estimated heart-beat rate and the first estimated heart-beat rate is less than a threshold while a difference between the second estimated heart-beat rate and the third estimated heart-beat rate is less than the threshold, the heart-beat rate is an average of the first estimated heart-beat rate, the second estimated heart-beat rate, and the third estimated heart-beat rate.

17. The physiological signal sensor in claim 16, wherein if the difference between the second estimated heart-beat rate and the first estimated heart-beat rate is less than the threshold while the difference between the second estimated heart-beat rate and the third estimated heart-beat rate is not less than the threshold, the heart-beat rate is an average of the first estimated heart-beat rate and the second estimated heart-beat rate.

18. The physiological signal sensor in claim 16, wherein if the difference between the second estimated heart-beat rate and the first estimated heart-beat rate is not less than the threshold while the difference between the second estimated heart-beat rate and the third estimated heart-beat rate is less than the threshold, the heart-beat rate is an average of the second estimated heart-beat rate and the third estimated heart-beat rate.

19. The physiological signal sensor in claim 16, wherein if the difference between the second estimated heart-beat rate and the first estimated heart-beat rate is not less than the threshold while the difference between the second estimated heart-beat rate and the third estimated heart-beat rate is not less than the threshold, the heart-beat rate is the second estimated heart-beat rate.

20. The physiological signal sensor in claim 1, wherein the first processor performs a de-resonance algorithm for calculating the respiration rate.
